(19) United States
(12) Patent Application Publication
(10) Pub. No.: US 2021/0285035 A1
(43) Pub. Date: Sep. 16, 2021

(54) SINGLE MOLECULE CONTROLS

(71) Applicant: DNAe Group Holdings Limited, London (GB)

(72) Inventor: Brian McKeown, Chipping Norton Oxfordshire (GB)

(21) Appl. No.: 15/779,274

(22) PCT Filed: Nov. 23, 2016
(86) PCT No.: PCT/GB2016/053649

§ 371 Date: Aug. 29, 2018

(30) Foreign Application Priority Data
Nov. 26, 2015 (GB) 1520883.8
Mar. 24, 2016 (GB) 1605055.1

(51) Int. Cl. C12Q 1/6834; C12Q 1/6851

(52) U.S. Cl. CPC C12Q 1/6834; C12Q 1/6851

(57) ABSTRACT

Methods for obtaining a reaction volume having a predetermined copy number, such as a single copy, of a known nucleic acid molecule therein are disclosed. Such a reaction volume may be useful as a control for nucleic acid amplification reactions. Nucleic acid constructs useful in such methods are also described. The constructs include a first region and a second region, wherein the second region is flanked by first and second primer binding sites to allow amplification across the second region, and wherein a selectively cleavable region is located between the first and second regions, with the selectively cleavable region being flanked by third and fourth primer binding sites to allow amplification across the selectively cleavable region. The second region acts as a reporter to indicate the presence of the entire nucleic acid molecule; and the selectively cleavable region can be cleaved to separate the first region from the second region, leaving an isolated copy of the first region.

### FIELD OF THE INVENTION

The present invention relates to methods for obtaining a reaction volume having a predetermined copy number of a known nucleic acid molecule therein. Such a reaction volume may be useful as a control for nucleic acid amplification reactions. Aspects of the invention relate to a nucleic acid molecule that may be useful in such methods. Further aspects of the invention are described herein.

### BACKGROUND OF THE INVENTION

Molecular Diagnostic Testing is the process by which the presence (or absence) of a certain DNA sequence (or other molecular species, such as a protein) is confirmed with high sensitivity and specificity. Sensitivity is the ability to detect the analyte even when there are physically very few copies of the analyte. Specificity is the ability to reliably detect the analyte whilst failing to erroneously detect any other species present, which may only be marginally distinct from the analyte of interest. The desirable combination of sensitivity and specificity is achieved when a rare species is detected with great veracity whilst present in a test sample in which many other very similar molecular species are present. High sensitivity and specificity is often hard to achieve and are critical considerations as to whether a technology is judged to be fit for purpose or not. A false negative may be generated by a test that has low sensitivity (regardless of how specific the test), whereas a false positive may be the result of a test that has low specificity, even if a high sensitivity test is used to detect an analyte that is absent from the test sample.

Molecular Diagnostic Testing is frequently employed during medical investigations, and the consequences of a false negative result, or a false positive result, can be detrimental to clinical decision-making and the selection of appropriate treatment for a patient.

It should be the goal of any sufficiently optimised molecular diagnostic test to return results that are reliable and reproducible, performing within set parameters. Such assurance is often obtained through the analysis of control samples concurrently with the test samples. These control samples are of known content, and therefore the outcome of the molecular tests performed upon these controls is predictable. Failure to generate the anticipated result from known controls, be they positive or negative, invalidates any results generated from concurrently run test samples. Good controls are consciously designed to be more prone to failure than the actual test sample, such that in the event of a positive result being generated from test sample, this is credible if the positive control has also generated a positive result. A good positive control is, by design, more likely to fail under standard test conditions than a genuinely positive test sample. This may be due to, for example, the control containing fewer representations of the analyte than are present in the test sample, putting greater demands of sensitivity on the control than the test sample. The provision of a meaningful control regime is key to the assembly of a well-designed and reliable molecular diagnostic test.

In the field of molecular diagnostics, there is a need to accurately test DNA analytes present in very low copy number, which may be as low as a single molecule of DNA, in a test sample that may be as complicated as a human genome. In situations where the test analyte (DNA sequence) is present at low single digit copies, and there may be a large number of other (similar) DNA species present, there is a need to provide a low copy number positive control that will validate the generation of a test sample positive result and, to some extent, validate the generation of a test sample negative result.

Controls, and specifically the means of generation disclosed here, are of broad utility to demonstrate the sensitivity of many amplification-based reactions and may, for example, be of utility in forensic science, demonstrating the efficiency of detection of a particular forensic target using a particular forensic DNA amplification technology.

Many other applications will also be enabled by the provision of controls that are of known numbers, especially single molecules. It is in the field of medical investigation that the greatest application of the invention is anticipated, for example in the detection of Blood Stream Infections or in the analysis of somatic mutations driving oncogenesis.

Among the objects of aspects of the invention is the provision of an artificial and manipulable control DNA analyte that can be co-amplified with a test sample, employing test components (DNA primers, for example) that are identical to those that interrogate the test sample for the presence of the test analyte. Because the control DNA analyte is amplified at the same time as the test analyte (if present), the detection of the control DNA analyte provides a reliable verification that the test, as assembled, was capable of amplification of the test analyte, even where that test analyte is itself only present at very low copy number, and perhaps as low as a single copy. However, the sensitivity of the system also provides some degree of verification of a negative test result: if the artificially introduced single copy control is detected with great certainty, the characteristics of the test design give some degree of confidence that a negative test sample result is indeed due to there being fewer than one molecule of the test analyte in the test sample, and that the test sample was therefore truly devoid of the test analyte.

### SUMMARY OF THE INVENTION

According to a first aspect of the invention, there is provided a nucleic acid cassette comprising a nucleic acid molecule comprising a first region and a second region, wherein the second region is flanked by first and second primer binding sites to allow amplification across the second region, and wherein a selectively cleavable region is located between the first and second regions, with the selectively cleavable region being flanked by third and fourth primer binding sites to allow amplification across the selectively cleavable region.

As will be apparent from the detailed description below, this nucleic acid cassette allows a reaction volume to be prepared having a predetermined copy number of a nucleic acid molecule comprising the first region therein. In brief, though, the second region acts as a reporter to indicate the presence of the entire nucleic acid molecule (also termed a nucleic acid cassette); the selectively cleavable region can be cleaved to separate the first region from the second region, leaving an isolated copy of the first region. The second region may be termed herein a reporter region. The first region may be termed herein a mimic region (as it is intended to mimic a test sequence).

The first region may be flanked by fifth and sixth primer binding sites to allow amplification across the first region. In a preferred embodiment, the fifth and sixth primer binding sites correspond to primer binding sites flanking a desired test nucleic acid sequence. “Correspond to” means that a primer capable of binding to a given primer binding site in the nucleic acid molecule will also be capable of binding to the corresponding primer binding site in the test nucleic acid sequence. This means that the same primers may be used in an assay to amplify both the test sequence and the first (mimic) region. Hence the first region can act as a control to confirm that the nucleic acid amplification has been successful. In certain embodiments the fifth and sixth primer binding sites are identical to the corresponding primer binding sites, although in other embodiments the fifth and sixth primer binding sites are non-identical (for example, they may differ by 1, 2, 3, 4, 5, or more nucleotides) to the corresponding primer binding sites. Where the primer binding sites are non-identical, this reduces the efficiency of the nucleic acid amplification of the first region, which may be desirable when the first region is being used as a control.

The selectively cleavable region may be a native nuclease binding site; for example, a restriction enzyme site (which should not be found in the cassette other than in the cleavable region(s)). Synthetic nucleases, such as metallic complexes, or engineered nuclease activities such as CRISPR Cas9 and derivatives could also achieve directed strand breakage. Alternatively, the selectively cleavable region may be chemically cleavable or photo-cleavable, or a combination of the two; it may comprise modified nucleotides or a non-nucleotide moiety which can be selectively cleaved. For example O-nitrobenzyl modifications that render the nucleic acid photolabile, or a 7-nitro-indole modification, which permits light activation and subsequent mild alkaline or thermal cleavage.

The cassette may comprise multiple reporter regions, each flanked by primer binding sites. Each reporter region may be separated from its neighbour by a selectively cleavable region. The multiple reporter regions are preferably identical, as are the multiple selectively cleavable regions. In certain embodiments, however, the multiple reporter regions may be different.

The cassette may comprise multiple mimic regions. Each mimic region may be flanked by primer binding sites. In certain embodiments, the multiple mimic regions are different, while in others the multiple mimic regions are identical. Similarly, the multiple primer binding sites may be different, such that each mimic region may be amplified with a different primer pair.

Where the cassette comprises multiple reporter and/or mimic regions, these need not be in any particular order. For example, all reporter regions may be grouped together, or they may alternate with mimic regions, or any other order may be used.

The nucleic acid molecule may be linear, or it may be circular such as a plasmid.

The mimic region may be selected to possess certain desired properties; for example, length, G/C composition, absence of repeat sequences, and/or capacity to form secondary structures. The desired properties may vary depending on the desired use of the nucleic acid molecule; for example, when used as a control, the length of the mimic region may be selected so as to be similar but not necessarily identical to the desired test analyte sequence, allowing both to be readily distinguished if both are amplified in an assay. The desired properties may also be selected so as to mimic the test analyte region, such that conditions which allow amplification of the control will also be expected to allow amplification of the test analyte sequence.

The reporter region may comprise modified nucleotides; for example, certain nucleotides may be labelled with a detectable label. This may aid its use as a reporter. Alternatively the reporter region need not be labelled, and can be detected in some other way; for example, by the use of dyes which detect dsDNA.

The nucleic acid molecule is preferably DNA.

The nucleic acid molecule may be immobilised on a solid support. The solid support may be a bead, a membrane, an adsorbent surface, or the like.

Also provided is a solid support having a nucleic acid cassette as herein defined immobilised thereon.

A further aspect of the invention provides a method for obtaining a predetermined copy number of a known nucleic acid molecule, the method comprising the steps of:
(a) preparing a plurality of reaction mix volumes containing a nucleic acid cassette as herein described such that at least some of said volumes are statistically likely to contain a desired predetermined copy number of said cassette;
(b) combining each of the plurality of volumes of (a) with an agent capable of cleaving the selectively cleavable region of the cassette, to thereby separate the first region and the second region;
(c) combining each of the plurality of volumes of (b) with nucleic acid primers capable of binding to the third and fourth primer binding sites flanking the selectively cleavable region of the cassette, and conducting an amplification reaction, to thereby amplify a sequence across the selectively cleavable region in those volumes where cleavage did not take place; and discarding those volumes where amplification took place;
(d) combining each of the remaining plurality of volumes of (c) with nucleic acid primers capable of binding to the first and second primer binding sites flanking the reporter region, and conducting an amplification reaction, to thereby amplify a sequence across the reporter region; and discarding those volumes where no amplification took place;
(e) to thereby provide a remaining plurality of volumes each of which comprises a predetermined copy number of a nucleic acid molecule comprising the first region.

In embodiments where the nucleic acid cassette comprises fifth and sixth primer binding sites flanking the first region, then the nucleic acid molecule in the plurality of volumes in (e) will also comprise the primer binding sites.

The plurality of reaction mix volumes of (a) may be prepared as a water-in-oil emulsion of an aqueous solution comprising the cassette. Each emulsion droplet represents a reaction volume. The emulsion is preferably prepared by combining an aqueous solution comprising the cassette with oil. The emulsion may be achieved by mixing, sonication, or injection of aqueous solution into oil. This latter is preferred as it allows greater control over droplet volumes. The droplets may be nano, pico, or femtolitre scale volumes. The emulsion may contain a detergent, to help maintain the droplets separate.

When preparing an emulsion, the desired copy number can be achieved by using a relatively low concentration of the cassette in the aqueous solution. The concentration can be selected such that this results in a statistical distribution within the volumes such that the majority of volumes will include no cassette, while at least some include the desired copy number of cassettes (and some may include more than the desired copy number).

Preferably, the desired copy number of cassettes is one.

The desired copy number of the nucleic acid molecule may be the same as the desired copy number of cassettes. Preferably this is one, and this may be achieved by use of a cassette having a single first region. Alternatively, the nucleic acid molecule copy number may be greater than the copy number of cassettes; copy numbers which are a multiple of the copy number of cassettes may be achieved using cassettes having more than one identical first region. For example, where the cassettes have two first regions, and the cassette copy number is one, then the copy number of the nucleic acid molecule will be two.

Preferably the majority of reaction mix volumes in (a) include no copies of the cassette. By “at least some” of the volumes containing the desired copy number of the cassette is meant at least 10, 9, 8, 7, 6, 5, 4, 3, 2, 1, 0.1, 0.01, 0.001% of the volumes contain the desired copy number.

One or more, and preferably all, of the combining steps may be carried out by fusion of two or more reaction mix volumes. These volumes may be in the form of droplets, for example, water-in-oil droplets. Fusion of droplets can be carried out using microfluidics systems, with known techniques. For example, suitable techniques include forced merging within a microchannel (described in Hung et al, Lab Chip, 2006, 6, 174-178) or collision between droplets through Electrowetting-on-Dielectric (EWOD) (described in Fan et al Lab Chip. 2009 May 7;9(9):1236-42). In alternative embodiments, the reaction mix volumes may be contained within a reaction vessel, for example, a multiwell plate. In these embodiments, reaction mixes may be successively added to a reaction vessel in order to combine them. Either approach allows for nucleic acid amplification to be carried out in situ, for example by thermal cycling reactions.

Nucleic acid amplification preferably comprises polymerase chain reaction (PCR) amplification. Necessary reagents for nucleic acid amplification may be combined with the reaction mix volumes at each step, or may be contained within the initial reaction mix volume, such that only the necessary primers are added at each step. Necessary reagents may include DNA polymerase enzyme, buffers, dNTPs, and so forth. The skilled person will be aware of how to perform nucleic acid amplification, and which reagents are necessary.

The nucleic acid amplifications may be labelled. This allows ready determination of whether amplification has occurred or not. Labelling may be performed with a dye, eg, a fluorescent dye, to quantitate the amount of nucleic acid in the reaction mix. For example, a dye may be used which binds to dsDNA (such as SYBR green). Different amplifications may use different dyes, to allow each amplification to be distinguished. Alternatively labelled nucleotides may be incorporated into each amplification.

Step (d) may further comprise quantitating the amplified reporter region, and discarding those reaction mixes where the amount of amplified nucleic acid is above and/or below a predetermined threshold. For example, this may be used to indicate that the starting copy number of the cassette was greater than the desired copy number—in such a case, the starting copy number of the reporter region will be higher than anticipated, and the final amount of amplified reporter will also be higher than anticipated.

The method may further comprise the step of binding the nucleic acid molecules comprising the first region contained in the volumes of (e) to a solid support. For example, the support may be a microbead, a membrane, an adsorbent material, etc. Each volume may be bound to a separate solid support, or a separate region of a solid support. In this way, a single (or a desired copy number) nucleic acid molecule may be bound to a solid support. The method may further comprise the step of combining the solid support with a reaction mix volume containing a test nucleic acid, and allowing the test nucleic acid to hybridise to the bound nucleic acid(s) on the solid support. In this way the bound nucleic acid molecule (of known copy number) may be used to isolate a test nucleic acid containing complementary sequences. Importantly, this will be isolated at known copy number. Alternatively, the nucleic acid molecules comprising the first region may be adsorbed into a solid support (for example, a hydrophilic and lipophobic material, eg, a cellulose-based filter paper). The use of a hydrophilic and lipophobic material allows the aqueous phase of a water-in-oil droplet containing the nucleic acid to be adsorbed (preferably in its entirety) into the material, while the oil phase is not adsorbed, and can be removed.

The method may further comprise the step of combining the volumes obtained in (e) with a reaction mix volume containing a test nucleic acid and a primer pair which will bind to the fifth and sixth primer binding sites; performing nucleic acid amplification on the combined reaction mix; and determining whether nucleic acid amplification has taken place of i) the nucleic acid molecules comprising the first region; and/or ii) a portion of the test nucleic acid. The first region thereby acts as a control to indicate that the amplification reaction is proceeding.

The method may further comprise the step of combining the volumes obtained in (e) with an additional reaction mix having different physical properties. For example, the additional reaction mix may have a greater volume, a greater viscosity, or both.

A further aspect of the invention provides a method for performing a nucleic acid assay to detect the presence of a test nucleic acid in a sample, the test nucleic acid being flanked by fifth and sixth primer binding sites corresponding to fifth and sixth primer binding sites flanking a first region of a nucleic acid molecule as defined above; the method comprising:
(a) combining a sample containing a test nucleic acid with a reaction volume prepared as described above, and with a primer pair which binds to the fifth and sixth primer binding sites;
(b) performing a nucleic acid amplification reaction on the combined sample and reaction volume;
(c) determining whether nucleic acid amplification has taken place of i) the nucleic acid molecules comprising the first region; and/or ii) a portion of the test nucleic acid.

A yet further aspect of the invention provides a solid support comprising a hydrophilic and lipophobic material having a nucleic acid molecule of known copy number adsorbed thereon.

A still further aspect of the invention provides a method for isolating a target nucleic acid molecule in a known copy number, the method comprising the steps of:
a) contacting i) a solid support having a nucleic acid molecule of known copy number attached thereto with ii) a solution comprising a plurality of nucleic acid molecules, at least one of which is a target nucleic acid molecule, wherein at least a portion of the target nucleic acid molecule is complementary to a portion of the nucleic acid molecule attached to the solid support;
b) allowing the nucleic acid molecule attached to the solid support to hybridise to the target nucleic acid molecule; and
c) removing the solid support from the solution;
to thereby isolate the hybridised target nucleic acid molecule in a known copy number.

The solid support having a nucleic acid molecule of known copy number attached thereto may be prepared as described herein, and as described with reference to the preceding aspects of the invention. The nucleic acid molecule of known copy number may be prepared as described with reference to the preceding aspects of the invention.

The nucleic acid molecule of known copy number may be double stranded or single stranded. Where double stranded, the method may further comprise the step of denaturing the double stranded nucleic acid molecule to allow hybridisation. Further, where double stranded, preferably only a single strand of the double stranded molecule is attached to the solid support.

The complementary portions of the molecules are preferably at least 10, 15, 20, 25, 30, 35, or 40 nucleotides in length. The complementary portions are preferably at least 85%, 90%, 95%, 97%, 99%, or 100% complementary.

Preferably at least a portion of the nucleic acid molecule of known copy number is not complementary to a corresponding portion of the target nucleic acid molecule. That is, both molecules have complementary portions, and the portions of the molecules adjacent these are not complementary, such that the molecules will not hybridise at the non-complementary portions. The non-complementary portion of the nucleic acid molecule of known copy number is preferably at the end of the molecule which is not attached to the solid support; preferably this is the 3′ end. There may also or instead be a further non-complementary portion of the molecule at the end which is attached to the solid support; this portion can be incorporated into any amplification products generated from the nucleic acid molecule of known copy number, and may be used for example to incorporate specific sequence tags or further binding sites.

Preferably the known copy number is 1.

Preferably the solution comprises multiple copies of the target nucleic acid molecule; more preferably there are significantly more copies of the target nucleic acid molecule than the known copy number.

The solid support is preferably a polymer bead.

Preferably a plurality of solid supports are provided, each having a nucleic acid molecule of known copy number attached thereto.

The method may further comprise the step d) delivering the solid support and the target nucleic acid molecule to a reaction vessel or a reaction volume. The reaction vessel may be a well. Preferably the well is dimensioned so as to be capable of receiving only a single solid support. The method may further or alternatively comprise the step e) extending the captured target nucleic acid molecule by a polymerisation reaction using the nucleic acid molecule of known copy number as a template, thereby incorporating additional sequence into the captured target nucleic acid molecule. The additional sequence is preferably a sequence which is not naturally found adjacent the captured target; this may be used, for example, to incorporate known primer binding sites or universal primer sites into the target molecule. The method may still further comprise the step f) amplifying at least a portion of the target nucleic acid molecule to provide a plurality of copies of the amplified portion.

### SUMMARY OF THE DRAWINGS

FIG. 1 represents a test nucleic acid, containing a target analyte sequence.

FIG. 2 shows a nucleic acid cassette.

FIG. 3 shows the nucleic acid cassette of FIG. 2 and primers which recognise primer binding sites in the cassette.

FIG. 4 shows the preparation of microdroplets of water in oil emulsion, containing the nucleic acid cassette of FIG. 2.

FIG. 5 shows the location of a block to amplification on the nucleic acid cassette.

FIG. 6 shows the location of the susceptible site on the nucleic acid cassette, and the use of primers to amplify across the site.

FIG. 7 shows the production of cleaved cassette.

FIG. 8 shows the attempted amplification across the susceptible site of the cassette.

FIG. 9 shows discrimination between droplets containing the desired sequence and those which do not.

FIG. 10 shows an overview of the method for obtaining a reaction volume containing a known copy number of a known nucleic acid sequence.

FIG. 11 shows different ways of manipulating the reaction mix droplet obtained as a result of the method.

FIG. 12 shows a diagnostic assay using the reaction volume.

FIG. 13 shows the result of amplifying the reaction volume.

FIG. 14 shows an alternative nucleic acid cassette.

FIG. 15 shows an alternative nucleic acid cassette.

FIG. 16 illustrates detection of multiple copies of a reporter sequence in a sample.

FIG. 17 shows an alternative nucleic acid cassette.

FIG. 18 is a schematic illustration of a nucleic acid molecule of known copy number being adsorbed into a solid support.

FIG. 19 shows a method of confirming the presence or absence of a control mimic nucleic acid sequence.

FIG. 20 illustrates attaching a control mimic nucleic acid molecule onto a solid support.

FIG. 21 shows an alternative nucleic acid cassette.

FIG. 22 shows a single molecule control mimic nucleic acid produced from the cassette of FIG. 21, hybridised to a complementary nucleic acid molecule.

FIG. 23 shows a polymer bead with a stably attached single strand of nucleic acid hybridised to a complementary strand of nucleic acid.

FIG. 24 illustrates a workflow that enables the recovery of a single nucleic acid strand from a plurality of nucleic acid strands, using the polymer bead of FIG. 23.

FIG. 25 shows a process to distinguish specifically hybridised complementary nucleic acid strands from non-specifically adsorbed nucleic acid strands.

FIG. 26 shows strand extension of a captured complementary recovered nucleic acid strand to incorporate novel sequence therein.

FIG. 27 depicts amplification of a captured complementary recovered nucleic acid strand and co-opting onto a surface for sequencing.

### DETAILED DESCRIPTION OF THE INVENTION

The present invention is intended to permit the generation of a reaction mix or reaction volume containing a controlled copy number (usually one) of a known and defined nucleic acid sequence. This volume may be used as a control in a molecular diagnostic assay. Although the invention is primarily described herein in terms of providing an artificial control sample, it will be appreciated that there are other fields where it may be desirable to generate a sample containing a known copy number of a known nucleic acid; the invention is thus not limited to generation of control sequences.

The sensitivity and specificity of DNA testing technologies has increased in recent years, such that it is now possible to detect a single molecule of a test analyte DNA sequence by a number of means, including clonal amplification of that test analyte DNA sequence and mass detection of the amplified product by, for example, Next Generation Sequence analysis.

However, the detection of single molecules (or very low single digit copies) is challenging. Any sufficiently developed assay must enjoy very high sensitivity and very high specificity, but frequently maximising one of these compromises the other. In the investigation of blood stream infection (BSI) for example, it may be clinically significant (and a patient may be very sick) where there is as low as 1 colony forming unit (CFU) of the pathogen per millilitre of whole blood. It is necessary to rapidly identify the pathogen and perhaps antimicrobial resistance (AMR) genes that might confer resistance to specific classes of antibiotic. With a minimal blood draw of 10 ml and acceptance that preparation of DNA from the pathogen will incur loss of a proportion of that DNA, it is not unreasonable to think that a molecular diagnostic test will have to be capable of accurately detecting low single digit copies of the target analyte in a background of large quantities (both physical and sequence-content) of ‘contaminating’ DNA, and reporting a result that can be relied upon by the clinician when selecting appropriate therapy.

The use of controls, run concomitantly with the test sample, has long been an accepted means of giving confidence that a diagnostic test is performing within the expected validation parameters. This is of particular relevance in demonstrating that the sensitivity of the assay is performing at the challenging levels of low single molecules in the test sample, but until now there has been no simple and reliable method that enables control samples to be generated to accurately reflect the very low copy number that might be encountered in a test sample.

The invention disclosed herein enables the provision of an artificial test sample that reliably mimics the very low copy number and the sequence context of a test analyte. Provision of such a control, which is co-amplified along with the test analyte, enables the performance of the assay to be confirmed, and the veracity of any test result generated to be confirmed. Even in situations where there is a negative test result, the veracity of this as a ‘true negative’ is confirmed to a greater extent where the control, run at a level of just a single molecule, does generate a convincing positive result.

Although amplification of very small quantities of DNA is prone to stochastic variability during early cycles, running a control that generates an output using the same amplification primer sequences as the test analyte, in the same tube as the test analyte, gives effective ‘normalisation’ of many experimental variables that might otherwise affect the efficiency of the test assay. Such variables as absolute concentration of reagents, pipetting errors and temperature fluctuations, plasticware and operator variables are automatically controlled for when the control and the test are run in the same reaction vessel. These variables can confound the usefulness of a control, if performed in a separate reaction vessel from the test assay. Other factors that can be designed to ensure that control and test sequences amplify with the same efficiency are for example the length of the amplicon, as there is a selective bias to amplify short amplicons with greater efficiency. The ‘GC’ content of the intervening sequence may also impact how efficiently sequences of identical length are amplified. These and other factors might be designed deliberately into the control, where it may be beneficial to make that control slightly ‘harder to amplify’, and therefore more prone to failure, as any good control should be. If designed to be as functionally equivalent as possible, then the primer binding sites, the length and the GC content and distribution may be normalised, together with other sequence considerations, such as runs of homopolymer and capacity to form secondary structures.

A detailed description of the invention is now given, for illustrative purposes only. In this example, the detection of a blood stream infection pathogen is used as an exemplar, in which it might be expected that any pathogen nucleic acids indicative of infection would likely be present at very low (single digit) copy number. The example also envisages the use of microdroplets as reaction volumes, and to contain the nucleic acid cassette; such microdroplets are amenable to manipulation and combination by using microfluidics techniques. Combination of a first droplet with additional reagents can be achieved relatively straightforwardly by fusion of two droplets. Again, however, the invention is not limited to the use of microdroplets.

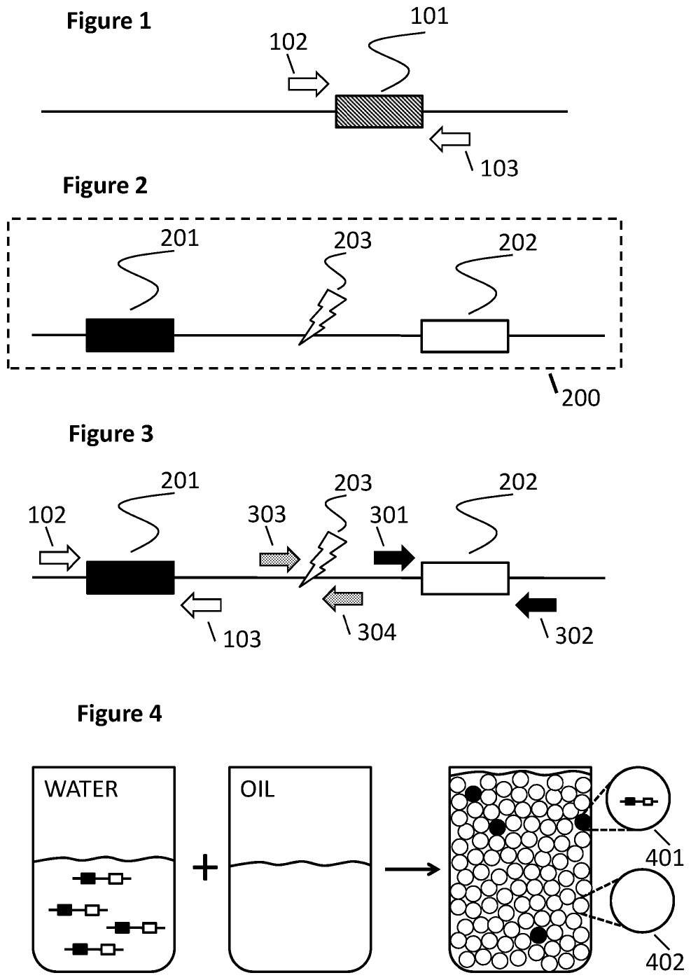

FIG. 1 shows a test nucleic acid. The section of DNA that is indicative of infection (and is the target of a molecular diagnostic test) is labelled as a grey hatched box 101. This test analyte 101 can be considered an informative marker, and is bounded or flanked by areas of DNA sequence that are specifically targeted by, for example, PCR primers that are represented as white block arrows 102 and 103. The PCR primers will bind to suitable primer binding sites within the test nucleic acid and (in the presence of suitable reagents such as DNA polymerase, nucleotides, and buffers) permit amplification of the region within the primers 102, 103. No further distinction is made as to forward 102 and reverse direction 103, save for the orientation of these block arrows; where the current document refers to “primers” or “primer binding sites” it will be understood that such primers or sites typically refer to a pair of such primers or sites flanking a sequence to be amplified. The design, selection, and use of primer pairs for amplification of target DNA is well understood, and is within the capability of the skilled person. Template DNA prepared from a clinical sample will be interrogated by a diagnostic assay to determine the presence (and perhaps semi-quantitative presence) or absence of this informative marker.

In order to provide a control for the diagnostic detection of the test analyte 101, an artificial nucleic acid construct is provided. This artificial construct is called the CONTROL CASSETTE 200, as shown in FIG. 2. The control cassette 200 is comprised of three covalently-linked, separate regions/features:
ANALYTE MIMIC 201
REPORTER 202
SUSCEPTIBLE SITE 203

Importantly, the susceptible site 203 is located between the analyte mimic 201 and the reporter 202. The susceptible site 203 could be, for example, a restriction endonuclease cleavage recognition sequence. The sequences of the analyte mimic 201 and the reporter 202 are not necessarily derived from any naturally occurring sequence, and therefore may be any optimally designed sequence. However, typically the analyte mimic 201 will be selected to have at least some similarity (for example, length, G/C content, etc) to the test analyte 101, but equally to be distinguishable therefrom when both sequences are amplified.

Whilst the DNA sequences of the analyte mimic 201 and reporter 202 are unconstrained in their selection, the DNA sequence of the susceptible site 203 and the sequences flanking all three of the regions 201, 202 and 203 are constrained such that specific amplification primers can be provided to hybridise to these sites. FIG. 3 shows that the three regions 201, 202 and 203 can be targeted using primers that are distinct from each other. Note however that the primers that target amplification of analyte mimic 201 are identical (or at least substantially identical) to the primers 102 and 103 capable of amplifying the test analyte 101 in FIG. 1. Therefore, in a sample in which the test analyte 101 and the analyte mimic 201 are both present, these distinct DNA sequences will both be amplified by the same forward primer 102 and reverse primer 103. In certain embodiments of the invention, amplification of the analyte mimic 201 may not be necessary; in such embodiments the presence of primer binding sites flanking the analyte mimic 201 and corresponding to primers 102 and 103 is not essential. For example, if the mimic 201 is not intended to be used as a control in an amplification reaction, but used for hybridisation to a desired target, then it is not essential to include primer binding sites.

Primers 301 and 302 are designed to amplify the reporter 202, and primers 303 and 304 are designed to amplify through the susceptible site 203 provided the nucleic acid cassette is intact. In certain embodiments, one or other or both of primers 303 and 304 may include 5′ tails of non-template nucleotides, to aid strand displacement of these primers when annealed. However, this is not believed to be essential, and in preferred embodiments the primers do not include tails.

Primers 102 and 103 are selected so as to allow hybridisation to both the cassette and the test sample, and are therefore based on DNA sequences found in the test sample. However, primers 301, 302, 303 and 304 are not necessarily derived from any naturally occurring sequences and can be freely optimised. In particular, primers 301, 302, 303, and 304 can be designed such that there is no (or minimal) unwanted hybridisation between these primers and the target sites of primers 102 and 103, or indeed these primers and their respective non-target sites. This design strategy reduces the chances of unintended amplification of analyte mimic 201; specifically, the primers should not be able to hybridise anywhere which could result in the unwanted duplication of the mimic region 201 since this could clearly compromise the ability to produce a single copy of that region in the final product. Likewise, the sequence of the mimic region 201 can also be designed so as to reduce or eliminate unwanted hybridisation. It will be understood, therefore, that only the sequences of the primers 102 and 103, and their respective binding sites, are constrained by the desired target sequence; other sequences may in certain embodiments be freely designed in order to optimise performance. In certain embodiments, the respective melting temperatures of a primer to its target may also or instead be designed to arrive at a preferred range of melting temperatures of different primer:target hybridisations. For example, primers 301 and 302 may have a lower melting temperature than primers 303 and 304. Such an arrangement may permit a further check against amplification of an unwanted region during performance of the present methods.

This invention provides a method whereby a single copy (or other known copy number) of the analyte mimic 201 may be delivered to a reaction volume, and which may therefore be used as a control in a diagnostic assay where the presence of the test analyte 101 is being assessed. The first step of this process is to distribute the control cassette 200 construct into isolated volumes, such as by formation of an emulsion of ‘water-in-oil’ droplets or other small volume reaction chambers. The format of ‘water-in-oil’ droplets will be used as an exemplar in FIG. 4 and henceforth.

A known concentration of the control cassette 200 is prepared in aqueous solution, such that when combined with oil, small volume (nano, pico or femtolitre range) droplets are created. The concentration of the control cassette in the initial solution is sufficiently low that the overwhelming majority of the ‘water-in-oil’ droplets formed will be entirely devoid of the control cassette 200; these droplets are identified in FIG. 4 as 402. A very small number of the droplets will however contain the control cassette 200, identified as 401, but virtually none of these 401 species will contain more than one copy of the control cassette 200. Using Poisson distribution, the majority of the 401 droplets will contain just one copy of the control cassette 200. Thus, statistically, at least some of the droplets will contain the desired copy number of the control cassette.

The concentration of the initial solution, and the volume of oil used, may be varied to achieve a desired final distribution of the cassette in the droplets.

There are a number of means that ‘water-in-oil’ droplets can be formed, including vigorous mixing, sonication or directly injecting the aqueous solution through a narrow constriction into the oil. This final method is preferred as it gives the greatest potential to control the uniform volume of the resulting droplets. Whichever method is chosen, the final result of the process is a very large number of droplets, maintained separately to each other (through, for example, the inclusion of a detergent within the aqueous solution).

The mixed population of droplets 401 and 402 must now be individually identified and separated, which is achieved by sequential utilisation of the susceptible site 203 and the reporter 202 that are linked to the analyte mimic 201 on the control cassette 200.

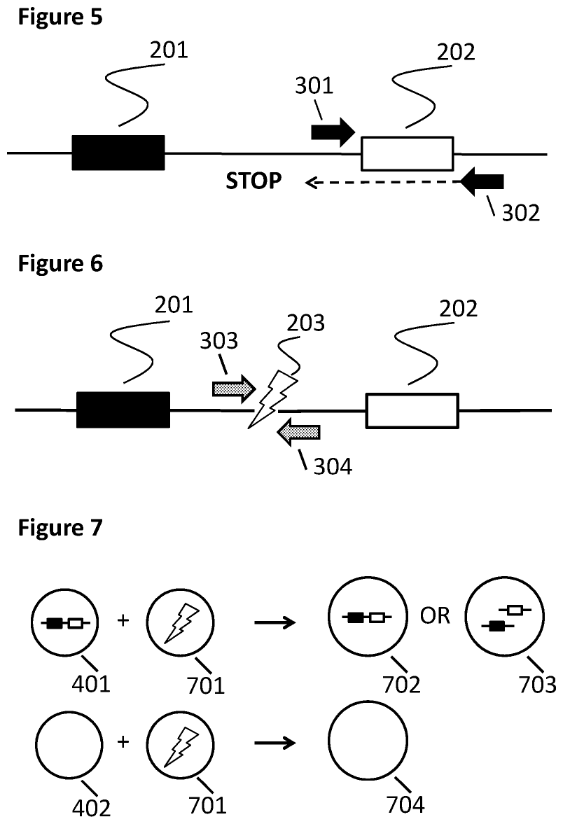

If the 401/402 population were to be analysed directly by, for example, amplifying the reporter 202, then this would risk unwanted amplification of the mimic 201 which is still on the same molecule as the reporter 202. For example, one possible method of confirming the presence of the reporter 202 would be to combine the individual droplets of the 401/402 mixture with a separate species of droplet enclosing biochemistry including primers 301 and 302, and subjecting the combined droplets to an amplification reaction. The presence of the reporter 202 could then be confirmed, through (for example) monitoring the accumulation of the reporter 202 amplicon. This can be achieved by including a dye that increases in fluorescence in the presence of increasing accumulation of dsDNA. However, if this is done on the intact cassette, there is the potential that the primer 302 would extend through the reporter 202, through the binding site for the primer 301, through the binding site for primer 304, through the intact susceptible site 203 and its primer site 303 and on through the region represented by the analyte mimic 201, and its associated primer binding sites 102 and 103. This undesirable duplication would mean that the analyte mimic 201 would no longer be present as a single copy within the droplet. This undesirable copying of the analyte mimic 201 would be linear (as opposed to exponential) and would only duplicate the analyte mimic 201 on one strand. Duplication of the analyte mimic 201 must therefore be prevented by introducing a physical blockage to the passage of the actively extending primer 302. FIG. 5 depicts the positioning of this blockade between the analyte mimic 201 and the reporter 202.

The nature of the blockade that would prevent the passage of the extending primer 302 could be, for example;
Hybridisation of a high-affinity 5′ phosphorylated (or other 5′-3′ exonuclease digestion-refractory modification) oligonucleotide to the strand-separated template DNA, necessarily prior to initiation of extension from the reverse primer 302;
Incorporation or generation of abasic sites;
Inclusion of ‘PCR stoppers’, such as HEG (hexaethylene glycol);
Generation of strand nicks or physical breaks in the DNA.

However, systems that rely on the hybridisation of blocking molecules cannot be guaranteed to be 100% effective, and systems that rely on the direct inclusion of abasic sites, HEG, or other chemical blockers mean the control DNA is no longer ‘natural’, and is less amenable to future manipulation, as might be necessary to introduce new control sequences for new targeted test analytes or to generate copies of the sequence for manufacturing purposes.

The present invention therefore makes use of a method whereby the mimic 201 and reporter 202 are physically separated prior to detection of the reporter 202. This ensures that amplification of the reporter 202 does not inadvertently also bring about the risk of amplification of the mimic 201. The most attractive and effective means of preventing the extension of reverse primer 302 through analyte mimic 201 during the polymerase-driven identification of the reporter 202 would be to physically break the covalent linkage of the analyte mimic 201 and reporter 202, necessarily after the intact control cassette 200 has been delivered to a specific droplet 401. This physical breakage can be achieved by the inclusion of the susceptible site 203 between analyte mimic 201 and the reporter 202 (but confirmed as being absent from any other regions within the control cassette 200). Restriction enzyme digestion of the cassette 200 using an enzyme which recognises or cuts at the susceptible site 203 will cleave the cassette 200 into two parts. As the susceptible site 203 is flanked by DNA sequences that can support hybridisation of oligonucleotide primers 303 and 304, then amplification across this region can be used to confirm whether cleavage has taken place or not. See FIG. 6.

Initially, in order to determine which of the droplets within the mixture of 401 and 402 droplets do indeed contain the control cassette 200, each one of these droplets must be treated as if it does contain this construct and attempts to inactivate the susceptible site 203 must be made (by restriction digestion, for example). The droplets contained within mixture 401 and 402 are therefore individually merged with a further species of droplet, 701, that contains the necessary biochemistry to effect inactivation (cleavage) of the susceptible site 203, as shown in FIG. 7. Each of the merged droplets is incubated such that the inactivation biochemistry has adequate opportunity to inactivate the susceptible site 203, should that site be present. In this way, droplet 401 (which did contain the control cassette 200) will give rise to two possible droplet species; droplet 702 in which inactivation of the susceptible site 203 has been ineffective and the analyte mimic 201 and reporter 202 remain linked, and droplet 703 in which the inactivation has been effective and the analyte mimic 201 and reporter 202 are separated from each other. Droplet 402, which did not contain the control cassette 200 gives rise to a droplet species 704. Droplet 704 will be the majority species. Note that in droplet 703, although the analyte mimic 201 and the reporter 202 are no longer associated by functional linkage, both these sequences are present in 1:1 molar ratio, and most likely as single copies of the two sequences.

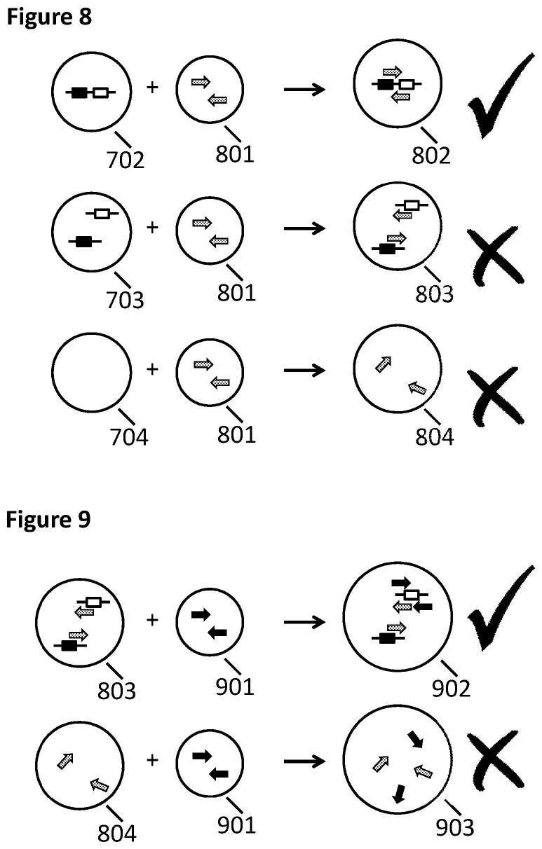

In order to discriminate droplets 702, 703 and 704, polymerisation across the susceptible site 203 with primers 303 and 304 is effected (see FIG. 8). This requires merger of every individual droplet 702, 703 and 704 with a droplet species 801 that contains biochemistry capable of effecting amplification across the susceptible site 203, if still intact. The droplet 801 may include, for example, polymerase, nucleotides, buffers, and a dsDNA dye such as SYBR Green fluorescent dye to allow monitoring of accumulation of amplicon. In some embodiments of the invention, some components of the biochemistry may be already present in the originally-generated droplets 401, 402 (for example, by inclusion of polymerase and nucleotides, but not primers, in the starting solution of the control cassette). Droplet species 802, 803 and 804 are thermal cycled in order to allow DNA polymerisation to occur, and real time, or fixed-point/end-point, fluorescent assessment of the individual droplets is performed. Positive amplification, assessed as an increase in the fluorescence within a droplet 802, is indicative of an intact susceptible site 203, and identification of that droplet as derived from species 702. Both droplets 703 and 704 will fail to generate an increase in fluorescence upon thermal cycling with primers 303 and 304, and both of these species 803 and 804 can be taken forward for further analysis. Species 802 is separated to waste.

Note that it is preferable to initially assess the inactivation of the susceptible site 203, rather than identifying the (relatively small number of) droplets that contained the reporter 202 region first. This is because assessing reporter 202 followed by susceptible site 203 would necessitate discrimination of a positive fluorescent result building on a previously positive fluorescent result. Although it would not be impossible, it would be clearer and more preferred to have a positive result generated (from assessment of reporter 202) subsequent to a negative result (from failure to amplify across the susceptible site 203). This strategy however demands assessment of a very great number of species 704 droplets, the majority of droplet species devoid of the control cassette 200.

In certain embodiments of the invention, however, it may be possible to assess the reporter 202 first, if two different dyes (for example, fluorescing at different wavelengths) were used to assess the reporter 202 and susceptible site 203. This might be achieved for example by using TaqMan probes, which generate fluorescence after cleavage of a probe during Taq polymerase extension. TaqMan probes could be targeted against the reporter (Wavelength 1 positive result indicative of presence) and then the susceptible site 203 (Wavelength 2 failure to generate a positive response indicative of successful cleavage of the susceptible site 203).

Droplets 803 and 804, which failed to amplify across the susceptible site 203, must now be discriminated through assessment of the presence of the reporter 202. These droplets are individually merged with droplet species 901 (see FIG. 9), which contains necessary biochemistry (for example, polymerase, nucleotides, etc) and primers 301 and 302, potentially with additional SYBR Green dsDNA dye. FIG. 9 demonstrates the merger of these droplets. Droplet 902 (containing the reporter) will support amplification of the reporter 202, although this fragment of the control cassette may also support (ineffectual) hybridisation of the susceptible site 203 reverse primer 304, carried over from earlier stages of the analysis. However, as noted above the primer binding sites may be optimised to prevent or reduce primer “crosstalk” or unwanted hybridisation. In this case, the primer binding sites are chosen so that primer 304 (susceptible site primer) and primer 302 (reporter 202 forward primer) do not overlap, and as such do not interfere with each other. Similarly, the primer 303 (susceptible site 203 forward primer) will carry over from earlier analysis, and could ineffectually hybridise to the analyte mimic 201, although again, this primer is designed so that it does not overlap with primer 103 (analyte mimic 201 reverse primer).

Once merged, droplets 902 and 903 are thermal cycled under conditions that will amplify the reporter 202 region, if present. Only droplet 902 will demonstrate this amplification and generate an increase in fluorescence as the SYBR Green dye binds to the amplicon generated.

Droplet 902 has therefore been generated from a progenitor droplet that has sequentially;
Failed to demonstrate an intact susceptible site 203
Positively demonstrated the presence of reporter 202

This droplet 902 is therefore confidently determined to include a nucleic acid molecule comprising the analyte mimic 201 and flanking primer binding sites, which has been separated from the reporter 202 by cleavage of the susceptible site 203. This droplet will contain a single copy of the analyte mimic 201 (in the majority of cases), due to the low starting concentration and distribution within the original droplets of the control cassette 200, and will also contain the detritus of the various analyses used to confirm its identity. Given that this droplet can be manipulated into a diagnostic assay that seeks to confirm the presence of the test analyte 101 by virtue of amplification with primers 102 and 103, and that the carried over biochemistry droplet 902 harbours does not interfere with the performance of primers 102 and 103, this droplet 902 provides a single copy analyte mimic 201, amenable to amplification by primers 102 and 103.

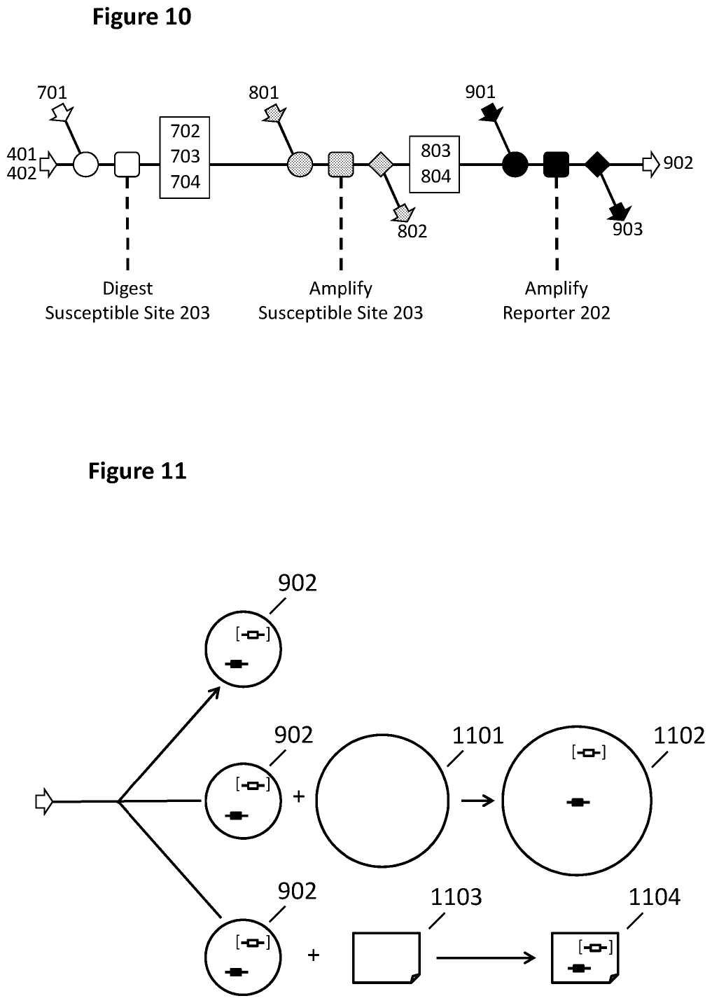

An overview of the flow of droplets, mergers and rejections is given in FIG. 10. Droplets 401 and 402 (the starting population of droplets, some of which contain the control cassette 200) are individually merged with droplet 701, which provides biochemistry to cleave the susceptible site 203. All droplets so created (702, 703 and 704) are incubated, within the white box, for a given time such that the inactivation of the susceptible site 203 is complete. All droplets 702, 703 and 704 are then flowed onwards where each is individually merged with a droplet 801, within the grey circle, creating droplets 802, 803 and 804. Droplet 801 provides biochemistry to amplify across the susceptible site 203 when thermal cycled within the grey box. After thermal cycling, the droplets 802, 803 and 804 are assessed (grey diamond), identifying 802 as fluorescent due to amplification across the uncleaved susceptible site, and rejected. Droplets 803 and 804, which have not demonstrated amplification and hence either contain a cleaved susceptible site 203 or no control cassette, are flowed onwards. Droplets 803 and 804 are individually merged with droplet 901, within the black circle. Droplet 901 provides biochemistry to amplify the reporter 202 within the black box.

After thermal cycling, the droplet 902 is identified (within the black diamond) as being fluorescent due to reporter amplification, and is harvested as the final product of the process. Droplet 903, which remains non-fluorescent and therefore devoid of the reporter 202, is discarded to waste.

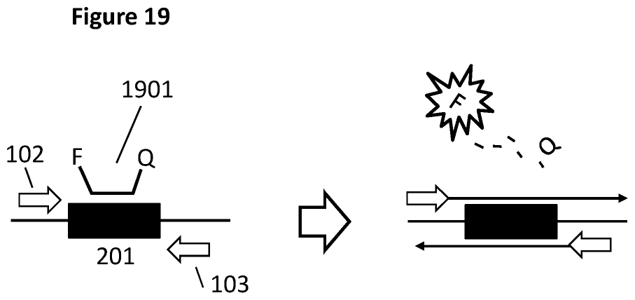

As a final demonstration of the effectiveness of this scheme, provision of a TaqMan probe designed to hybridise to the control mimic 201 sequence and biochemistry to amplify this region (primers 102 and 103) can be used to distinguish droplet 902 from droplets 903: only species 902 should support a positive TaqMan reaction, with the fluorescent emission of the TaqMan reaction being by necessity in a different channel to that of the dye already used to confirm the presence of the reporter 202 (SybrGreen for example). This confirmatory test is depicted in FIG. 19, but is a demonstration of the success of the scheme only: this confirmation clearly relies on amplification of the 201 control mimic sequence within droplet 902, eliminating its ‘single molecule’ credentials.

During manufacture, no TaqMan assay will be performed to confirm the presence of control mimic 201; the control mimic 201 will remain as a single molecule. Modulating the physical nature of droplet 902 is desirable in order to enable it to be manipulated, such that its contents are easily introduced into a diagnostic assay. FIG. 11 demonstrates three alternatives for this manipulation, although there will be many others. Option 1; it may be possible to deliver the droplet 902 contents directly to the reaction chamber of the ultimate diagnostic assay. Option 2: the physical nature of droplet 902 may be changed by merging it with another droplet 1101 (represented as being physically greater volume, but perhaps also or instead a higher viscosity/solidity) or some other characteristic that allows the merged droplet 1102 to be ‘handled’ (that is, easily further manipulated such as pipetted or flowed into an additional reaction chamber or vessel). Option 3 shows a preferred embodiment, where the entire contents of the aqueous droplet 902 are adsorbed into a lipophobic, hydrophilic solid support matrix 1103 that holds the single copy of the analyte mimic 201 in a stable format, and permits the (possibly dried) product 1104 to be manipulated into the diagnostic assay, within which the adsorbed single copy of analyte mimic 201 is available to the biochemistry of that assay. The solid support matrix 1103 may for example comprise a cellulose or cellulose-based matrix, and so forth. A schematic of this approach is shown in FIG. 18, which illustrates, from left to right, a droplet containing the single copy nucleic acid molecule passing along a channel having a pore therein. Adjacent the pore is a cellulose matrix; as can be seen from the Figure the droplet is adsorbed in its entirety into the matrix (through a combination of capillarity and amenable hydrophilicity) and the nucleic acid retained thereon. As the matrix is lipophobic, any oils from the water-in-oil emulsion will not be adsorbed and will remain within the channel. This is further aided by the greater viscosity of the oil, which will retard exit through the pore. This matrix is easily handled and transported.

Further possible manipulations might include desiccation of the droplets to form rehydrateable pellets, with each pellet containing just a single representation of the analyte mimic 201. It might be beneficial to remove certain (or all) of the detritus of previous analyses from the final desired single molecule droplet, in case these components interfere with the ultimate molecular diagnostic test, although in preferred embodiments, these carry-over components are tolerated in the final diagnostic assay and need not be removed.

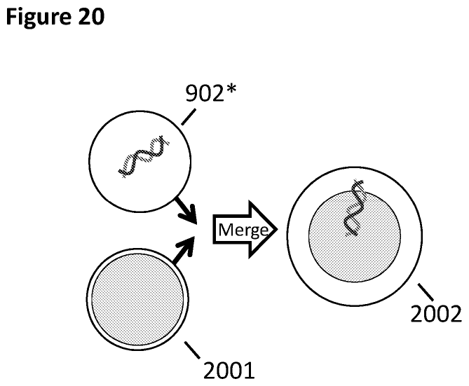

In other embodiments, the single copy nucleic acid could be bound to a solid support (rather than being simply adsorbed); for example, the solid support could be a polystyrene bead, a derivatised glass surface, etc. This may allow alternative uses of the nucleic acid, such as acting as a nucleic acid probe. In certain embodiments of the invention, such solid supports may be useful in isolating a single copy of another nucleic acid to which the single copy nucleic acid may hybridise. The single copy nucleic acid can act as a molecular “hook” or “fishing rod” and placed in a reaction mix containing a desired target; the target will hybridise to the hook, while the solid support allows the hybridised nucleic acids to be manipulated thereafter. Further details of the use of a single copy nucleic acid as a “fishing rod” are described elsewhere in this document, with reference to FIGS. 20 to 27.

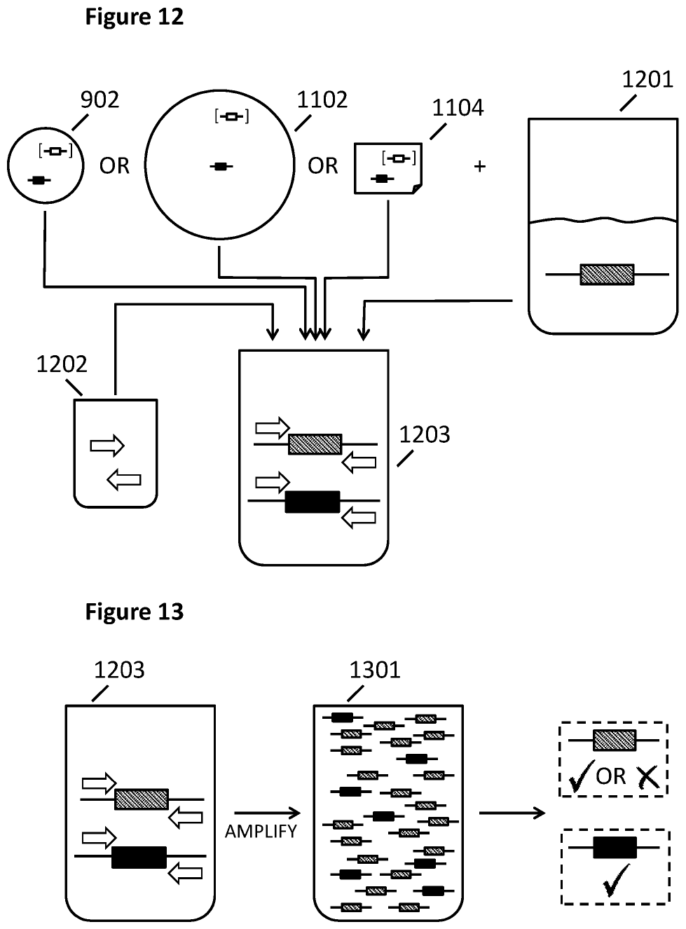

The diagnostic assay could be performed in a number of reaction vessels (as opposed to droplet-based water-in-oil), but is disclosed here as being a PCR amplification based system: the assay would require the combination of extracted test sample (Template DNA), the biochemistry required to amplify the test analyte 101, and the analyte mimic (in one of at least three formats disclosed here). This combination is represented diagrammatically in FIG. 12.

Droplet 902, ‘modified’ droplet 1102 or matrix 1104 will each harbour a single copy of the analyte mimic 201. When one (and only one) of these species is combined with test template 1201 and the biochemistry 1202 to support amplification of the test analyte 101/analyte mimic 201, the reaction vessel 1203 will be capable of simultaneous amplification of the test analyte 101 (grey hatched box) and the analyte mimic 201 (black box). Subsequent detection/discrimination of the amplified species by any suitable means allows the anticipated positive control analyte mimic 201 to inform the significance of a positive or negative result generated from the test analyte 101.

After amplification within the vessel 1203 by, for example, thermal cycling, the vessel may contain multiple copies of the test analyte 101 and the analyte mimic 201. These amplicon species will have common sequence at their terminal ends (by virtue of having both been generated through the extension of primers 102 and 103), but will have distinct core sequences. FIG. 13 demonstrates that post-amplification, there may be many copies of each species. It is anticipated that there will be multiple copies of the analyte mimic 201 species present, but the absolute number of these will be a function of stochastic fluctuations during the early cycles of amplification. Therefore, the consideration of numbers of copies of the analyte mimic 201 amplicon and the test analyte 101 amplicon will be, at best, semi-quantitative. This stochastic fluctuation is compounded by the potential that the test analyte 101 itself may be present in very low copy number and perhaps as low copy number as a single representation. Only if the test analyte 101 is absent from the test sample 1201 will the result be, in some sense, quantitative.

Irrespective of the presence or absence of the test analyte 101 in the vessel 1203, the analyte mimic 201 is confidently present and should be amplified to give multiple copies in the vessel 1301. If the test analyte 101 is also present this will amplify, but if it is absent, it will of course fail to amplify. The comparison of the number of copies of the analyte mimic 201 amplicon and the test analyte 101 may give some impression of the relative abundance of the test analyte 101 in the test sample, but this comparison will be at best semi-quantitative.

FIG. 19 depicts a final confirmation that the droplets, or other constrained reaction volumes, identified as species 902 do indeed contain a copy of the Control Mimic 201, and that droplet species 903 is indeed devoid of this Control Mimic 201. Through fusion of one final droplet species, or other delivery of biochemistry, a ‘TaqMan’ probe 1901 designed to hybridise within the Control Mimic 201 sequence will, upon amplification with primers 102 and 103, generate a fluorescent signal as the probe fluorophore (F) and quencher (Q) are detached from each other due to the 5′ to 3′ exonuclease activity of the DNA polymerase during PCR. The fluorophore attached to the TaqMan probe is specifically selected to be distinct (spectral emission) from the fluorophore of the dsDNA binding dye used to initially discriminate species 902 from species 903. This is necessary as droplet species 902 will already be fluorescent, due to the successful amplification and detection of the Reporter 202 region. Monitoring and detection of the fluorescent response of the fluorophore F in droplet species 902 exclusively confirms that the detection system correctly identifies species 902 droplets. Of course, this final confirmation amplifies the Control Mimic 201 sequence, and is only used to verify the production schema: it would not be routinely employed during production of droplet species 902 and isolation of the Control Mimic 902 at very low, possibly single copy, representations.

Enhancements of the system are envisaged that allow the system to be used to control for more than one test analyte in a multiplex molecular diagnostic test analysis. FIG. 14 demonstrates the inclusion of an additional analyte mimic 1401 within an alternative control cassette 1400, which still includes a single reporter 202. Note that the two discrete analyte mimics 201 and 1401 are separated from each other, and from the reporter 202 by identical (common) susceptible sites 203 (each of which is flanked by common primer binding sites, not shown here). If susceptible to cleavage by restriction digestion, it will be the same restriction enzyme that will effect the cleavage of the control cassette into three portions at the sites indicated in FIG. 14. Each analyte mimic 201, 1401 is flanked by distinct primer binding sites corresponding to those flanking the test analytes which each mimic is mimicking. Thus, each analyte mimic 201, 1401 can be separately amplified by using the appropriate primer pair.

Total digestion of this cassette 1400 at both the susceptible sites 203 is necessary to demonstrate that duplication of one or other or both of the analyte mimics 201 and 1401 has been prevented. Detection of an intact susceptible site 203 post-inactivation (by amplification across the site 203) cannot reveal which of the susceptible sites 203 has remained intact since they are identical. Regardless, an intact site 203 will result in that droplet being discarded. It may be that in certain embodiments only the susceptible site 203 located between the analyte mimic 1401 and the reporter 202 is absolutely required to prevent duplication of the analyte mimics during analysis of the reporter 202 (and hence the two analyte mimics 201, 1401 are not separated by a susceptible site, and will not be separated in the final droplet). However, for certainty, and to prevent confusion of amplicons where there are primers amplifying both analyte mimics, it is preferable to position a susceptible site 203 between the analyte mimics 201 and 1401.

A further enhancement of the system (shown in FIG. 15) is provided in order to more certainly ensure that there is just a single copy of the control cassette 200 present in droplet 401. This enhancement aims to limit the stochastic variability that is probable during early cycles of amplification of the reporter 202 by including multiple copies of this reporter 202. FIG. 15 shows a control cassette 1500, with the inclusion of two identical copies of the reporter 202, each separated from its neighbour by a susceptible site 203. Amplification of this double reporter 202 is more likely to generate a response that is quantifiable, and the greater the number of representations of the reporter 202 present at the beginning of the analysis, the less likely it is that stochastic effects will impact the ability to deliver a quantifiable result. Whereas the original control cassette 200, with its single representation of reporter 202, would produce a response of magnitude N upon analysis of the reporter 202, two copies of this control cassette 200 would generate a response of magnitude 2N. The same scenario where control cassette 1500 is used would generate responses of 2N (single copy of the cassette) and 4N (two copies of the cassette) and the difference is therefore of a greater magnitude and hence more readily discriminable. This step change in the response may be more quantifiable at a fixed-point during analysis (as opposed to end-point) and enable greater confidence in identifying the presence of a single copy of the control cassette over multiple copies.

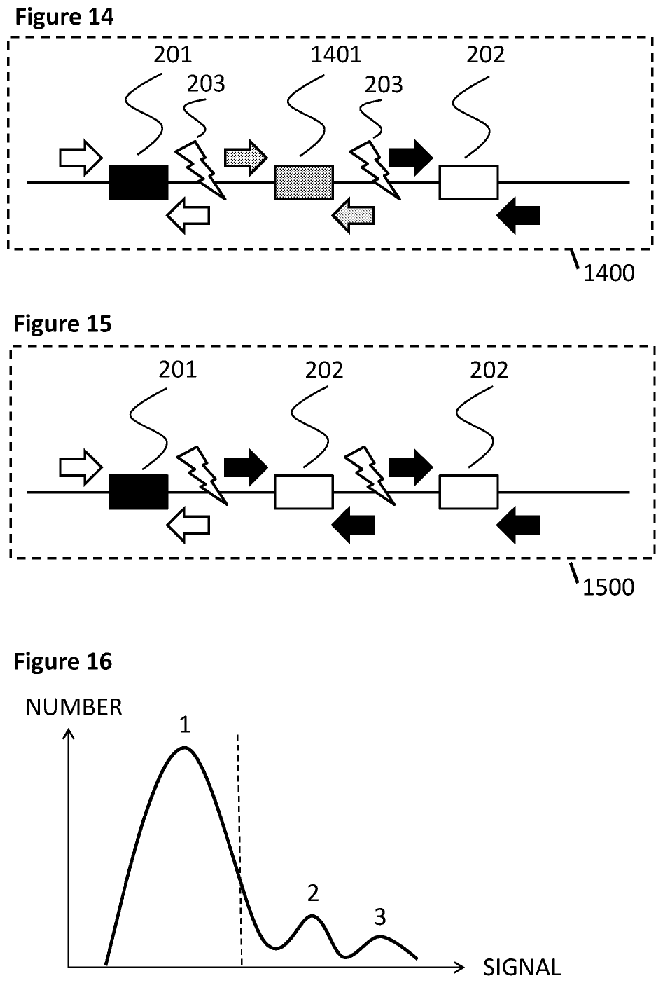

Comparison of the fixed-point (as opposed to end-point) responses from different representations of droplet 401 will enable the distribution of responses to be considered, and the potential for a secondary separation of the response to eliminate any overlap in the response, where the separation of the highest responses, together with any in a grey area, can be eliminated. FIG. 16 graphically depicts the distribution of the responses that might be seen. If multiple copies of the control cassette 200 (or 1500) are present within the droplet, this will boost the intensity of the signal returned after a limited degree of amplification (perhaps mid-point, rather than the ‘normalised’ end-point). Due to the stochastic nature of the amplification, it is possible that the peaks associated with 1, 2 and 3 (and greater) copies of the control cassette being present within the droplet will overlap, and as such, the dotted line in the FIG. 16 indicates the level at which all droplet 401s demonstrating greater signal than this level will be rejected as containing more than a single representation of the control cassette. Note that these occurrences will be rare, and it may be that there are insufficient copies of these droplet 401 to enable the generation of a bell-shaped curve, as depicted here. This situation will be all the more extreme for droplets containing 3 or more control cassette representations.

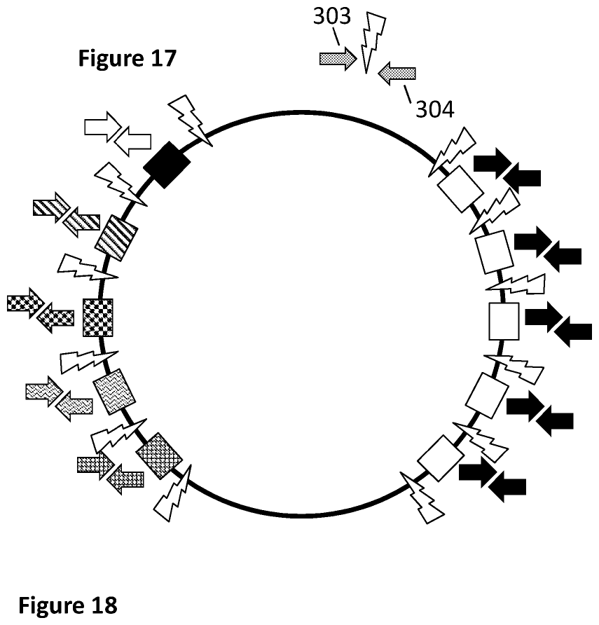

A final enhancement of the system, shown in FIG. 17, is the provision of multiple, distinct analyte mimics 201 and the provision of multiple, identical reporter 202 regions on a single cassette. This might be on a circular DNA system, such as a plasmid, as represented in FIG. 17. The provision of multiple analyte mimics (single copies) allows the same droplet 902 to be used in the multiplex analysis of several different test analytes 101 in the same assay. The provision of a much greater number of copies of reporters 202 is also indicated, and the greater the number, the clearer the distinction may be between the presence of a single copy of the control cassette and the presence of more than one, or multiple, copies. The example construct in FIG. 17 harbours single copies of 5 distinct analyte mimics 201 (shown by different shading) where each of these is flanked by a (identical) susceptible sites 203 and (distinct) primer binding sites that are derived from the appropriate test analytes 101. The control cassette also demonstrates 5 identical reporters 202 (white boxes) that are similarly flanked by susceptible sites 203 and by (identical) primer binding sites (black block arrows, 301 and 302). As shown, each of the susceptible sites (203) is flanked by primer binding sites 303 and 304 (grey block arrows). This arrangement is flexible, and can accommodate additional analyte mimics 201 as desired and additional reporter 202 regions as might be required.

The above detailed description demonstrates the flexibility and reliability of the invention to provide a system that can be used to provide a single copy of a mimic of the analyte in a format that enables the detection of the analyte to be assessed.

Although the illustrated embodiments have been described from the perspective of preparation of droplets, the use of droplets per se may not be required, as there exist methods of carrying out ‘digital PCR’ that rely on very small reaction chambers (nanolitre wells; eg Wafergen or Life Technologies QuantStudio DX) to which additional components could be sequentially added. For example, after the Poisson distribution of the original diluted control cassette 200 into the wells of a digital PCR chip, the susceptible site 203 inactivation biochemistry, the susceptible site 203 amplification biochemistry and the reporter 202 amplification biochemistry could be introduced to these wells. However, this is not preferred, as it is envisaged that recovery of the entire contents of the well after identification of those wells that contained a single copy of the control cassette could be very difficult. However, this approach may be useful where such recovery is not needed and further reactions performed in the same well. This sequential addition scheme may also be used to demonstrate the serial biochemistries are preforming as anticipated.

Another enhancement that might be desirable is that rather than assessing the degree of amplification of the reporter 202 sequence after a limited and quantifiable level of amplification, the individual droplets would most beneficially be amplified in a device which allowed massively parallel amplification and simultaneous real time PCR assessment, perhaps allowing more reliable quantification of the presence of the reporter 202 region.

As noted above, single copy nucleic acid sequences may be attached to beads or other solid supports in order to be used as molecular “hooks” or “fishing rods”. The following section describes this in more detail.

### CONTROLLED COPY NUMBERS AS SINGLE MOLECULE CAPTURE VEHICLES

Beyond the use as a sensitive control system, one attractive application of the ability to reliably isolate a single molecule of nucleic acid is the potential to use this (in its single stranded form) as a species-specific molecular ‘fishing hook’. If linked to a solid surface, for example a polymer bead, then the specific hybridization capacity of the single linked DNA sequence enables recovery of a second single DNA strand (bearing the complementary sequence) from a solution containing a plurality of DNA strands. The plurality of DNA strands may all bear the complementary sequence, or only a proportion of the plurality of DNA strands may bear the complementary sequence. Ideally, in order to maximize the potential for the bead-linked single molecule to encounter and capture a complementary DNA strand within a reasonable timeframe, the number of DNA strands in solution bearing the complementary sequence will be in massive excess compared to the single molecule linked to the bead. The complementary sequences can be designed to most advantageously allow high specificity, with little or no potential for cross-talk hybridization of the bead-linked capture sequence with any other DNA strand sequence that may exist within the plurality of DNA strands. Once captured, the DNA strand recovered from solution can then be manipulated as a non-covalently attached ‘passenger’ on the polymer bead.

The above system may be advantageously employed to seed the geographically-separated, clonal amplification of individual molecules (second single DNA strands) as the initiating step of an NGS reaction. Having delivered just one non-covalently attached passenger DNA strand to a specific geographical region, multiple (clonal) copies of identical sequence can be generated. Synchronous NGS interrogation of the bases of these copies maximizes the signal output generated at each individual base position of the DNA strand.

Delivery of a single copy of the DNA strand to be sequenced is ensured by virtue of the capacity to capture just one DNA strand onto each bead, and the geometric capacity to accommodate just a single polymer bead at each discrete geographic location. For example, after capture of a single DNA strand from a plurality of DNA strands, the bead harbouring the DNA strand may be delivered to a discrete well structure, where the well has dimensions sufficiently large to accommodate a single bead, but insufficiently large to accommodate more than one bead. This geometric limitation ensures that there will only ever be a single bead loaded to the well, and it follows only a single passenger DNA strand loaded per well. For example, such a system is described in WO2014/013263, to DNA Electronics Ltd. Reference is made particularly to page 15, line 25 to page 16, line 12; and to page 39, line 6 to page 43, line 15. These passages describe methods and systems for obtaining a limited number of beads per well, and for using such beads complexed with nucleic acids for sequence amplification and/or sequencing.

It is possible that non-specific adsorption of DNA stands from the plurality of DNA strands in solution onto the surface of the polymer bead may confound the aspiration to ensure just a single strand (passenger) of DNA delivered to the geographically distinct regions of the sequencing system. This potential may be minimized through stringent post-capture washing and/or selection of polymer bead/surface chemistry treatments that limit any such non-specific adsorption. Furthermore, it is possible to discriminate between legitimately captured DNA stands (hybridization captured) and non-specifically adsorbed sequences, by virtue of only the former having a potentially DNA polymerase-extendable, hybridized 3′0H end; the capture sequence attached to the bead may be specifically designed to include a non-hybridizable (i.e. not employed in capture) element that, upon extension of a legitimately captured DNA strand 3′ end, will drive DNA polymerase mediated incorporation of the complementary sequence to the non-hybridizable element (not employed in capture) onto the 3′ end of the captured DNA strand. The legitimately captured DNA strand is unique in its capacity to incorporate this additional sequence, which can subsequently be utilized as an essential element of the clonal amplification strategy (below).

The system detailed above is now illuminated using diagrammatic representations.

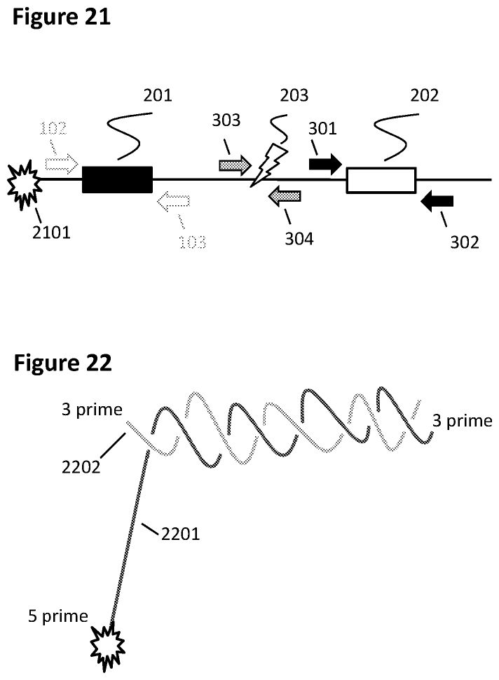

FIG. 20 continues the previously suggested manipulation of water in oil droplets, fusing the final ‘902 droplet’ species of FIG. 9 with a new droplet species containing a single reactive-surface polymer bead. A chemical reaction between the 5′ terminus of the single DNA molecule present in droplet 902* (‘*’, as the contents of this 902 species are chemically slightly different from before; see below) and the surface of the polymer bead contained within droplet 2001 is promoted, covalently linking the chemically active 5′ end of the (double stranded) single molecule of DNA contained in the 902* droplet. The bead/DNA hybrid so created within droplet 2002 is an article of manufacture and can be generated completely independently from its subsequent usage. FIG. 21 represents the previously described ‘Control Cassette’, enabling the identification and isolation of a single copy of a DNA sequence, 201. Whereas this sequence was previously flanked by binding sites for amplification primers 102 and 103 (here shown greyed out), which mimicked primers flanking some test analyte 101, there is no requirement in the ‘single molecule capture sequence’ embodiment to have this 201 sequence flanked by any specific amplification primer sequences, as this ‘capture sequence’ will never itself be amplified. Rather, the 201 capture sequence here acts as a ‘fishing hook’ that can be linked to a solid surface, such as a polymer bead. Therefore, this element of the cassette DNA is functionalized on the 5′ end of one strand (destined to remain single copy) with chemistry that will enable covalent linkage to surface chemistry on the polymer bead. This functionalization is represented in FIG. 21 by a starburst 2101. Possible functionalisations include but are not limited to amines, thiols and alkynes. Possible corresponding functionalization of the surface of the bead (or other solid surface) are NHS esters, maleimides or azide groups, but are not limited to these chemistries.

Once the Control Cassette is cleaved by restriction digestion (for example) and confirmed through the amplification of Reporter 202, the single molecule component 201 released may resemble the DNA element presented in FIG. 22. This is comprised of a reactive strand 2201 and a non-reactive strand 2202. The 5′ end of 2201 harbouring a reactive chemical modification need not be single stranded as represented here, but critically the light grey strand 2202 has no capacity to react with and become stably attached to the surface of a chemically reactive bead, and it can thus be washed away or otherwise removed after attachment of the darker reactive strand 2201. It may be beneficial to tolerate the persistence of the 2202 strand however, in order to promote stability of the essential, attached strand 2201.

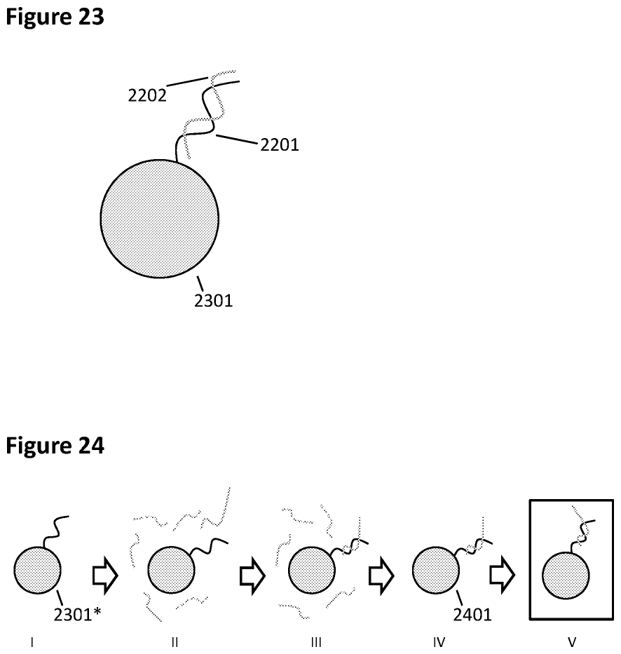

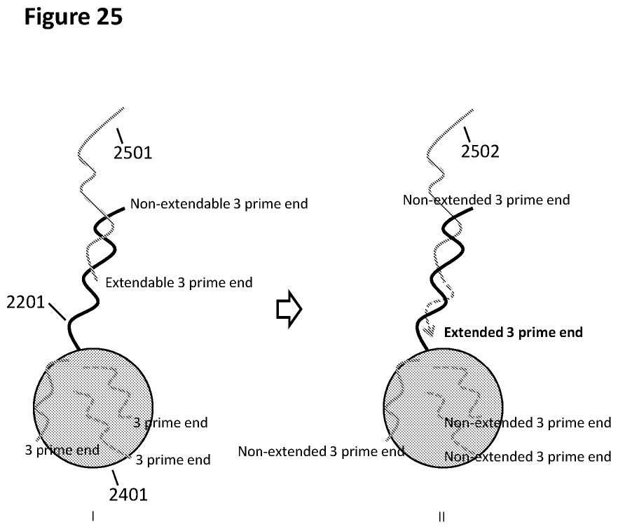

FIG. 23 illustrates one embodiment of the article of manufacture 2301: a polymer bead with a stably attached single strand of nucleic acid 2201. Greater integrity of the attached 2201 strand may be promoted by tolerating the persistence of the complementary 2202 strand (light grey line) until denaturation of the dsDNA attached to the bead/DNA hybrid, in the presence of the plurality of DNA strands harbouring the complementary sequence to 2201; competition will favour hybridization of the bead-linked 2201 capture sequence with a single member of the plurality of DNA strands than with the originally hybridized 2202 remnant (light grey line). After dissociation, the attached nucleic acid exposes a specific but artificial (i.e. not necessarily related to any naturally occurring nucleic acid) sequence. Ideally, this 2301 article of manufacture will be generated in massive numbers and be stable for a protracted period of time at ambient temperature. Advantageously, the 3′ end of the attached 2201 single stranded nucleic acid will not contribute to capture of a single strand of DNA from the plurality of DNA strands, but will be intentionally non-complementary over a small number of bases, sufficient to thwart DNA polymerase-mediated incorporation of nucleotides to this 3′ end (see also FIG. 25).

FIG. 24 illustrates a workflow that enables the recovery of a single DNA strand from a plurality of DNA strands and its subsequent delivery into a constrained geographic location. The article of manufacture 2301* (I) is represented as a polymer bead with a single molecule of (single stranded in this ‘*’ representation) nucleic acid with a specific hybridization capacity. This bead is combined with a plurality of DNA strands (II) in denaturing conditions (elevated temperature, for example) such that the bead and attached single-stranded DNA are present in a ‘soup’ of single stranded DNA, a proportion of which may bear a sequence that is at least partially, and ideally entirely and discriminatorily, complementary to the 2201 nucleic acid attached to the bead (solid grey lines) and a proportion of which may not bear this complementary sequence (dashed grey lines). When the denaturing conditions are relaxed (lower temperature, for example) this promotes dsDNA formation (III) and the single stranded nucleic acids form double stranded associations based on their degree of complementarity. Given that there is a sufficient excess of DNA strands harbouring a DNA sequence at least partially, and ideally entirely and discriminatorily, complementary to the nucleic acid sequence attached to the bead, this bead-sequence will advantageously hybridise to one (and only one) of the plurality of DNA strands present. After this specific hybridization, all other nucleic acids, whether dsDNA, ssDNA or some hybrid forms can be removed by washing of the bead (IV) with appropriate stringency to remove the non-attached nucleic acids, but without disrupting the association of the single recovered DNA strand from the bead-attached sequence. Finally, the 2401 bead, (covalently attached capture sequence and hybridized single molecule of captured DNA) are deposited into a geographically isolated, size constricted location (V), where the hybridized single molecule can be clonally amplified to generate sufficient identical copies to support clear simultaneously NGS base interrogation.

FIG. 25 depicts a means by which only the legitimately hybridized DNA strands captured from a plurality of DNA strands can be differentiated from any DNA strand captured onto the bead through non-specific adsorption. At least some of these non-specifically adsorbed DNA strands may harbour a sequence that is at least partially complementary to the sequence 2201 attached to the bead. In FIG. 25(I), there is one legitimately captured strand 2501 (solid light grey line, from the plurality of strands of FIG. 24) attached to the black, bead linked Capture Strand 2201. This captured strand 2501 has an engaged 3′0H end, which is labelled as being an ‘Extendable 3′ end’; this is the ONLY extendable 3′ end in this image, as the 3′ end of the 2201 Capture Sequence is, by design, not extendable. This is most easy achieved by ensuring non-complementarity between the 3′ end of 2201 and the region of the captured strand 2501 adjacent to the to the captured region, but may also be achieved by the placement of, for example, HEG spacers or other species not entertained by DNA polymerase during PCR. Other strands of DNA that may have become non-specifically associated with the bead surface are shown as light grey solid or dashed lines, and specifically, these sequences will (even in the unlikely event of their extension) fail to incorporate a complementary sequence to that of the DNA Capture Sequence 2201, proximal to the surface of the bead and not involved in the initial hybridisation capture of the 2501 molecule from the plurality of DNA fragments. This sequence (complementary to the DNA Capture Sequence proximal to the bead) is shown as an arrow-headed dashed grey line extension, added onto the Extendable 3′ end of the Captured Strand in FIG. 25(II). This added sequence can only efficiently be attached to the legitimately captured strand 2501, forming species 2502, and this added sequence can hereafter be employed as an essential attribute of the ‘single molecule capture’ system during clonal amplification of this legitimately captured ‘single molecule’.

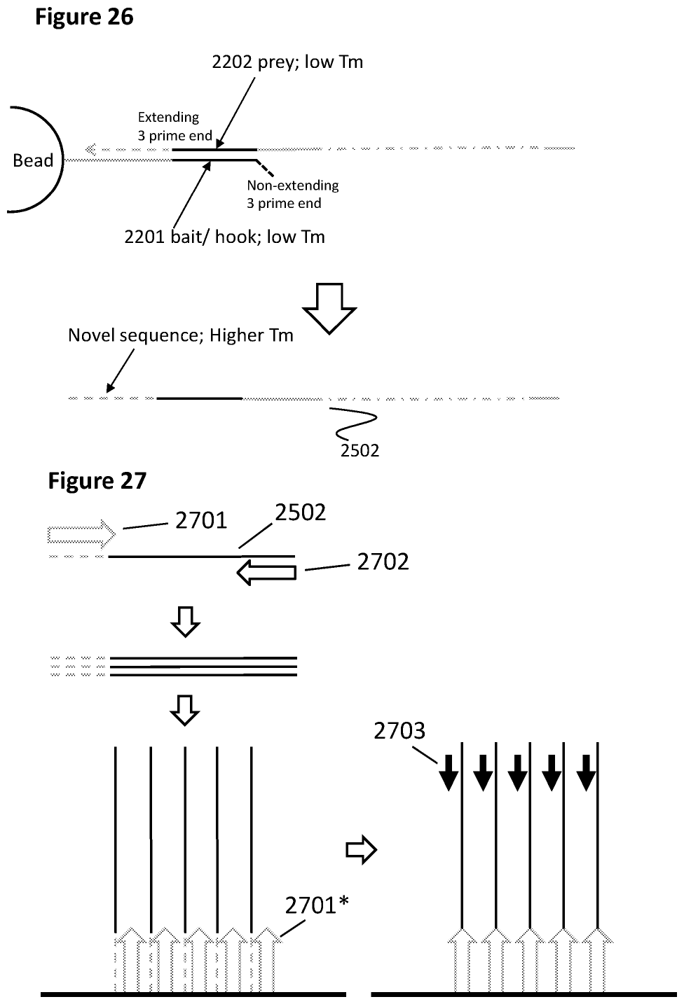

FIG. 26 depicts that when the Captured (2202 prey) sequence, for example a member of a library of nucleic acid sequences is hybridized to the Capture (2201 bait/hook) sequence, the hybridized 3′ end of the Captured sequence 2202 can be extended, yielding a product 2502 that is not covalently attached to the bead and that includes a novel 3′ end that is the complement of the bead attached capture sequence 2201, proximal to the bead. This novel 3′ sequence is now available to participate as an essential element during clonal amplification of the library molecule and permit amplification of the single captured library representative only, enabling NGS analysis of the light grey dot-dash area, for example using generic sequencing primers. Advantageously, the capture and DNA Polymerase extension of the hybridized 3′ end of 2202 is carried out at relatively low Tm. This is to ensure that only the correctly hybridized 2202 will be extended, as by elevating the Tm in subsequent rounds of amplification, the association between the capture sequence (bait) and the captured sequence (prey) is unfavourable, minimizing the potential for any remaining non-specifically associated strands that may be present upon the bead to replace the original, legitimately hybridized 2202 molecule, and support extension of these. Such replacement would potentially allow the essential sequence introduced to the 3′ end of the legitimate 2501 onto an interloper 2501 species, defeating the subsequent amplification of just the one molecule captured from the plurality of DNA strands during amplification. Selecting the Tm of capture (and initial extension (low Tm) to discriminate the Tm of subsequent clonal amplification of the novel 3′ end sequence (high Tm) ensures that this potential is minimized.

FIG. 27 depicts that amplification of the 2502 single molecule of (bead capture and 3′ extended; dotted grey portion) library molecule using a grey block arrow primer 2701 that will only amplify the products of FIG. 26 that have legitimately hybridized. Using an opposing primer 2702 (black block arrow) that hybridizes to a generic sequence introduced during PCR driven targeted library generation, the single molecule 2502 can be efficiently amplified within the constraints of a geographic region (a micro well, for example). Once sufficient solution phase amplification has occurred, the product of this solution phase amplification will be entertained by primers 2701*, which are attached on a surface, such that these surface attached primers (substantially or completely identical to the grey block arrow 2701 of the in-solution phase amplification) will extend and create a copy of the library amplicon that is physically attached to the surface of the geographically constrained region. During the PCR driven creation of the original library fragments, a generic sequence providing a sequencing primer hybridisation zone may have been incorporated, and this sequence is now available for the attachment of a sequencing primer 2703 and the performance of simultaneous NGS reactions upon many clonally amplified target molecules within one specific geographically constrained region, and simultaneously upon many separate geographically constrained regions (each containing copies of a different original library molecule) using the same generic sequencing primer 2703.

1. A nucleic acid cassette comprising a nucleic acid molecule comprising a first region and a second region, wherein the second region is flanked by first and second primer binding sites to allow amplification across the second region, and wherein a selectively cleavable region is located between the first and second regions, with the selectively cleavable region being flanked by third and fourth primer binding sites to allow amplification across the selectively cleavable region.

2. The nucleic acid cassette of claim 1 wherein the first region is flanked by fifth and sixth primer binding sites to allow amplification across the first region.

3. The nucleic acid cassette of claim 2 wherein the fifth and sixth primer binding sites correspond to primer binding sites flanking a desired test nucleic acid.

4. The nucleic acid cassette of any preceding claim wherein the selectively cleavable region is a restriction enzyme binding and/or cleavage site.

5. The nucleic acid cassette of any preceding claim comprising multiple second regions, each flanked by primer binding sites.

6. The nucleic acid cassette of claim 5 wherein each second region is separated from its neighbour by a selectively cleavable region.

7. The nucleic acid cassette of claim 6 wherein each selectively cleavable region is identical.

8. The nucleic acid cassette of any preceding claim comprising multiple first regions.

9. The nucleic acid cassette of claim 8 wherein each of the multiple first regions is different.

10. The nucleic acid cassette of any preceding claim wherein the first region is a mimic region, with a sequence selected so as to have properties mimicking those of a desired test nucleic acid sequence.

11. The nucleic acid cassette of claim 10 wherein the properties mimicking those of a desired test nucleic acid sequence comprise one or more properties selected from the group consisting of length, G/C composition, absence of repeat sequences, and capacity to form secondary structures.

12. The nucleic acid cassette of any preceding claim wherein the second region is a reporter region.

13. The nucleic acid cassette of any preceding claim, wherein the cassette is functionalised with a reactive group in the vicinity of the first region, preferably at an end of the cassette.

14. The nucleic acid cassette of any preceding claim, wherein the cassette is immobilised on a solid support.

15. A reaction mix comprising the nucleic acid cassette of any preceding claim.

16. A method for obtaining a predetermined copy number of a known nucleic acid molecule, the method comprising the steps of:
(a) preparing a plurality of reaction mix volumes containing a nucleic acid cassette according to any of claims 1 to 13 such that at least some of said volumes are statistically likely to contain a desired predetermined copy number of said cassette;
(b) combining each of the plurality of volumes of (a) with an agent capable of cleaving the selectively cleavable region of the cassette, to thereby separate the first region and the second region;
(c) combining each of the plurality of volumes of (b) with nucleic acid primers capable of binding to the third and fourth primer binding sites flanking the selectively cleavable region of the cassette, and conducting an amplification reaction, to thereby amplify a sequence across the selectively cleavable region in those volumes where cleavage did not take place; and discarding those volumes where amplification took place;
(d) combining each of the remaining plurality of volumes of (c) with nucleic acid primers capable of binding to the first and second primer binding sites flanking the second region, and conducting an amplification reaction, to thereby amplify a sequence across the second region; and discarding those volumes where no amplification took place;
(e) to thereby provide a remaining plurality of volumes each of which comprises a predetermined copy number of a nucleic acid molecule comprising the first region.

17. The method of claim 16 wherein the plurality of reaction mix volumes of (a) are prepared as a water-in-oil emulsion of an aqueous solution comprising the cassette.

18. The method of claim 17 wherein the concentration of the aqueous solution is selected such that this results in a statistical distribution within the volumes such that the majority of volumes will include no cassette, while at least some include the desired copy number of cassettes.

19. The method of any of claims 16 to 18 wherein the desired copy number of the nucleic acid molecule is one.

20. The method of any of claims 16 to 19 wherein the majority of reaction mix volumes in (a) include no copies of the cassette.

21. The method of any of claims 16 to 20 wherein one or more, and preferably all, of the combining steps are carried out by fusion or combination of two or more reaction mix volumes.

22. The method of any of claims 16 to 21 wherein the reaction mix volumes are in the form of droplets.

23. The method of any of claims 16 to 22 wherein nucleic acid amplification is polymerase chain reaction (PCR) amplification.

24. The method of any of claims 16 to 23 wherein step (d) further comprises quantitating the amplified second region, and discarding those reaction mixes where the amount of amplified nucleic acid is above and/or below a predetermined threshold.

25. The method of any of claims 16 to 24 further comprising the step of binding the nucleic acid molecules comprising the first region contained in the volumes of (e) to a solid support.

26. The method of claim 25 further comprising the step of combining the solid support with a reaction mix volume containing a test nucleic acid, and allowing the test nucleic acid to hybridise to the bound nucleic acid(s) on the solid support.

27. The method of claim 25 further comprising the steps of:
a′) contacting i) the solid support having a nucleic acid molecule comprising the first region bound thereto with ii) a solution comprising a plurality of nucleic acid molecules, at least one of which is a target nucleic acid molecule, wherein at least a portion of the target nucleic acid molecule is complementary to a portion of the nucleic acid molecule attached to the solid support;
b′) allowing the nucleic acid molecule attached to the solid support to hybridise to the target nucleic acid molecule; and
c′) removing the solid support from the solution;
to thereby isolate the hybridised target nucleic acid molecule in a known copy number.

28. The method of claim 27 wherein the nucleic acid molecule comprising the first region is double stranded, and wherein only a single strand of the double stranded molecule is attached to the solid support.

29. The method of any of claim 27 or 28, wherein at least a portion of the nucleic acid molecule comprising the first region is not complementary to a corresponding portion of the target nucleic acid molecule.

30. The method of claim 29 wherein a non complementary portion of the nucleic acid molecule comprising the first region is at an end of the molecule which is not attached to the solid support.

31. The method of any of claims 27 to 30 wherein the solution comprises significantly more copies of the target nucleic acid molecule than the known copy number.

32. The method of any of claims 27 to 31 wherein the solid support is a polymer bead.

33. The method of any of claims 27 to 32 further comprising the step d′) delivering the solid support and the target nucleic acid molecule to a reaction vessel or a reaction volume.

34. The method of claim 33 further comprising the step e′) extending the captured target nucleic acid molecule by a polymerisation reaction using the nucleic acid molecule comprising the first region as a template, thereby incorporating additional sequence into the captured target nucleic acid molecule.

35. The method of claim 33 or 34 further comprising the step f′) amplifying at least a portion of the target nucleic acid molecule to provide a plurality of copies of the amplified portion.

36. The method of any of claims 16 to 24 further comprising the step of adsorbing the nucleic acid molecules comprising the first region contained in the volumes of (e) onto or into a solid support.

37. The method of claim 36 wherein the solid support is hydrophilic and lipophobic.

38. The method of claim 36 or 37 wherein the solid support is a cellulose-based matrix.

39. The method of any of claims 16 to 24 or 36 to 38, wherein the first region is flanked by fifth and sixth primer binding sites which correspond to primer binding sites flanking a desired test analyte nucleic acid sequence; the method further comprising the step of combining the volumes obtained in (e) with a reaction mix volume containing a test nucleic acid and a primer pair which will bind to the fifth and sixth primer binding sites;
performing nucleic acid amplification on the combined reaction mix; and determining whether nucleic acid amplification has taken place of i) the nucleic acid molecules comprising the first region; and/or ii) a portion of the test nucleic acid.

40. The method of any of claims 16 to 24 further comprising the step of combining the volumes obtained in (e) with an additional reaction mix having different physical properties.

41. A method for performing a nucleic acid assay to detect the presence of a test nucleic acid in a sample, the method comprising:
(a) combining a sample containing a test nucleic acid with a nucleic acid molecule of known copy number having a first region which is flanked by fifth and sixth primer binding sites which correspond to primer binding sites flanking a desired test nucleic acid sequence; and with a primer pair which binds to the fifth and sixth primer binding sites;
(b) performing a nucleic acid amplification reaction on the combined sample and reaction mix;
(c) determining whether nucleic acid amplification has taken place of i) the nucleic acid molecule comprising the first region; and/or ii) a portion of the test nucleic acid.

42. The method of claim 41, wherein the nucleic acid molecule of known copy number is prepared according to the method of any of claim 12 to 38 or 40.

43. The method of claim 41 or 42 wherein the known copy number is one.

44. A product comprising a hydrophilic and lipophobic solid support, having a nucleic acid molecule of known copy number adsorbed thereon.

45. The product of claim 44, wherein the solid support comprises a cellulose-based matrix.

46. The product of claim 44, wherein the solid support comprises a polymeric bead.

47. The product of claims 44 to 46, wherein the nucleic acid molecule comprises a first region flanked by fifth and sixth primer binding sites which correspond to primer binding sites flanking a desired test nucleic acid sequence.

48. The product of any one of claims 44 to 47, wherein the nucleic acid molecule of known copy number is prepared according to the method of any of claim 12 to 38 or 40.

49. A method for isolating a target nucleic acid molecule in a known copy number, the method comprising the steps of:
a) contacting i) a solid support having a nucleic acid molecule of known copy number attached thereto with ii) a solution comprising a plurality of nucleic acid molecules, at least one of which is a target nucleic acid molecule, wherein at least a portion of the target nucleic acid molecule is complementary to a portion of the nucleic acid molecule attached to the solid support;
b) allowing the nucleic acid molecule attached to the solid support to hybridise to the target nucleic acid molecule; and
c) removing the solid support from the solution;
to thereby isolate the hybridised target nucleic acid molecule in a known copy number.

50. The method of claim 49, wherein the solid support having a nucleic acid molecule of known copy number attached thereto is prepared in accordance with any of claims 14 or 25 to 38.

51. The method of any of claim 49 or 50 wherein the nucleic acid molecule of known copy number is double stranded, and wherein only a single strand of the double stranded molecule is attached to the solid support.

52. The method of any of claims 49 to 51, wherein at least a portion of the nucleic acid molecule of known copy number is not complementary to a corresponding portion of the target nucleic acid molecule.

53. The method of claim 52 wherein a non complementary portion of the nucleic acid molecule of known copy number is at an end of the molecule which is not attached to the solid support.

54. The method of claim 52 or 53 wherein a non complementary portion of the nucleic acid molecule of known copy number is at an end of the molecule which is attached to the solid support.

55. The method of any of claims 49 to 54 wherein the solution comprises significantly more copies of the target nucleic acid molecule than the known copy number.

56. The method of any of claims 49 to 55 wherein the solid support is a polymer bead.

57. The method of any of claims 49 to 56 further comprising the step d) delivering the solid support and the target nucleic acid molecule to a reaction vessel or a reaction volume.

58. The method of claim 57 further comprising the step e) extending the captured target nucleic acid molecule by a polymerisation reaction using the nucleic acid molecule of known copy number as a template, thereby incorporating additional sequence into the captured target nucleic acid molecule.

59. The method of claim 57 or 58 further comprising the step f) amplifying at least a portion of the target nucleic acid molecule to provide a plurality of copies of the amplified portion.
